(19) United States
(12) Patent Application Publication
(10) Pub. No.: US 2004/0137532 A1
(43) Pub. Date: Jul. 15, 2004

(54) Antibody competition test (act) for the qualitative and conformation dependent quantitative detection of the conformation changes of p-glycoprotein and other cell surface receptors, pumps and proteins, and uses thereof

(76) Inventors: Gabor Szabo, Debrecen (HU); Gyorgy Lustyik, Pecs (HU); Henrietta Nagy, Debrecen (HU); Katalin Goda, Debrecen (HU)

Correspondence Address: Doran R Pace, Saliwanchik Lloyd & Saliwanchik, A Professional Association, 2421 NW 41st Street Suite A 1, Gainesville, FL, 32606-6669, US

(21) Appl. No.: 10/469,720

(22) PCT Filed: Mar. 1, 2002
(86) PCT No.: PCT/HU02/00025

(30) Foreign Application Priority Data
Mar. 2, 2001 (HU) P 0100948

(51) Int. Cl. G01N 33/53; G01N 33/567

(52) U.S. Cl. 435/7.2

(57) ABSTRACT

The invention relates to the detection of the presence and/or conformation change of the cell surface receptors, pumps or proteins, when two different antibodies and optionally ligands are bound to the receptors, pumps and/or proteins recognized by the antibodies, and the change in the antibody-binding is measured. The method can be used for the determination of cytostatic drug combination, effective in case of multidrug resistance wherein the native cells are combined in vitro with the drug or drug combination to be tested, and the sample is tested with the method mentioned above. The method can be used for the detection of the efficiency of multidrug-resistance modulators wherein the method according is carried out in the presence of known modulator or a modulator to be tested, and the resulting activity values are compared. Also the object of the invention is a diagnostic kit for the detection of the presence and conformation change of receptors, pumps and proteins, for the determination of the efficient drug combination in case of multidrug resistance and/or for the detection of the efficiency of the modulators comprising a first antibody which is able to detect the receptors, pumps or proteins, optionally other ligands, and a system, which provides a system for the detection of binding.

### TECHNICAL FIELD

The present invention relates to an antibody competition test (ACT) for the qualitative and conformation-dependent quantitative detection of the conformation changes of P-glycoprotein and other cell surface receptors, pumps and proteins, and uses thereof. More detailed, the object of the present invention is preferably an antibody competition test, “ACT”, which can be used for the detection of the conformation changes following the receptor binding of a protein, P-glycoprotein (Pgp), frequently responsible for the multi-drug-resistance, or the ligand binding of the CD4 receptor, involved in the HIV infection. According to the invention the ratio of the binding of two antibodies or other specific protein-binding substance, suitably fluorescently labelled with two different colour stains, is measured, and from the changes of this ratio, by the effect of other ligands, the conformation changes of the protein is concluded. The two antibodies, differently labelled with two stains, can be specific to the same, or overlapping, or allosterically coupled epitopes. The invention refers to those cases, where the binding of the two antibodies (labelled ligand) is not changing to the same degree or in the same direction by the conformation effects of the receptor/pump/protein, generated by some kind of ligand-binding. On the one hand, the change of this ratio reflects more exactly the changes in the conformation than the change of the sole binding of any antibody in itself, on the other hand, the relative degree of the simultaneous binding of the two antibody can be strongly conformation dependent.

The essence of a fundamental embodiment of the invention is summarized in the following figure:

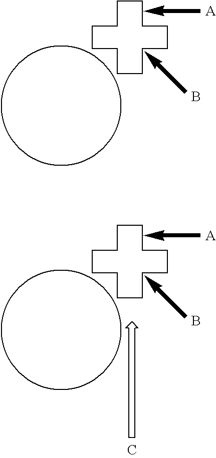

Two antibodies (“A” and “B”), binding to a cell surface receptor (here: pump), in the presence of a third ligand (“C”), bind in a modified ratio. The cross sign means antigen; the oval sign means cell.

Two antibodies or ligands, binding to the same receptor, bind to a given protein in proportion to their applied concentrations or their affinities (K_{assoc}). The latter parameters can change if the the conformation of the protein changes. This change of the K_{assoc}, which can be interpreted to the antibodies or to the ligand, is generally small, its detection is unreliable and it is not sufficiently sensitive. If the two antibodies or ligands bind to the same, or to overlapping or to allosterically coupled receptors or epitopes, then the increase in the binding of one of them can be accompanied by the decrease of the other. In these cases the simultaneous measurement of the two binding can result in the very exact and very sensitive detectability of the conformation change.

### BACKGROUND ART

Tumors are frequently resistant against chemotherapy, in such a way, that the intracellular concentration of the chemotherapeutic agents falls under the toxic level. The ABC (ATP Binding Cassette) transporter, a P-glycoprotein (Pgp), which is frequently responsible for the Multi Drug Resistance (MDR) symptom, is able to remove wide spectrum of the cytostatics from the cells, defeating the chemotherapeutic therapy [Gottesman M M. Pastan I: Biochemistry of multidrug resistance mediated by the multidrug transporter, Annu. Rev. Biochem. 62, 385-427 (1993)]. Special feature of Pgp that it can transport many amphiphile-lipophlic substances, which structurally are not similar too much to each other, and their size is changing in a wide range [Gottesman M M. Pastan I: Biochemistry of multidrug resistance mediated by the multidrug transporter, Annu. Rev. Biochem. 62, 385-427 (1993); Gottesman, M. M., Hrycyna, C. A., Schoenlein, P. V., Germann, U. A. and Pastan, I.: Genetic analysis of the multidrug transporter, Annu. Rev. Genet. 29, 607-649 (1995); Seelig, A: A general pattern for substrate recognition by P-glycoprotein, European Journal of Biochemistry 251, 252-2619 (1998)]. In approximately half of the human malignant tumours the Pgp is expressed on such a level, that in itself can cause clinically relevant drug-resistance [Gottesman M M. Pastan I: Biochemistry of multidrug resistance mediated by the multidrug transporter, Annu. Rev. Biochem. 62, 385-427 (1993); Gottesman, M. M., Hrycyna, C. A., Schoenlein, P. V., Germann, U. A. and Pastan, I.: Genetic analysis of the multidrug transporter, Annu. Rev. Genet. 29, 607-649 (1995); Nooter K. Sonneveld P.: Clinical Relevance of P-glycoprotein expression in haematological malignances, Leukemia Res., 18, 233-243 (1994); Bosch, I. and Crop, J.: P-glycoprotein multidrug resistance and cancer, Biochimica et Biophysica Acta 1288, 37-54 (1996); Sonneveld, P:: Prognostic significance and reversal of MDR in multiple myeloma, pp. 463-471, IN: Multidrug resistance in cancer cells. Molecular, biochemical, physiological and biological aspects, Ed.: Gupta S. and Tsuruo T., John Wiley and Sons (1996); Aran, J M., Pastan, I. and Gottesman, M M.: Therapeutic strategies involving the multidrug resistance phenotype: The MDR1 gene as target, chemoprotectant and selectable marker in gene therapy, Adv. Pharmacology 46, 1-42 (1999); Doige C. A. and Ames, G. F.: ATP-dependent transport systems in bacteria and humans: relevance to cystic fibrosis and multidrug resistance, Ann. Rev. Microbiol. 47, 291-319 (1993)]. It is a generally accepted theory, that the presence of the Pgp in the tumour cells provides a selection advantage during the progression of the tumour [Gottesman, M. M., Hrycyna, C. A., Schoenlein, P. V., Germann, U. A. and Pastan, I.: Genetic analysis of the multidrug transporter, Annu. Rev. Genet. 29, 607-649 (1995); Seelig, A: A general pattern for substrate recognition by P-glycoprotein, European Journal of Biochemistry 251, 252-2619 (1998); Bosch, I. and Crop, J.: P-glycoprotein multidrug resistance and cancer, Biochimica et Biophysica Acta 1288, 37-54 (1996); Sonneveld, P:: Prognostic significance and reversal of MDR in multiple myeloma, pp. 463-471, In: Multidrug resistance in cancer cells. Molecular, biochemical, physiological and biological aspects, Ed.: Gupta S. and Tsuruo T., John Wiley and Sons (1996); Aran, J M., Pastan, I. and Gottesman, M M.: Therapeutic strategies involving the multidrug resistance phenotype: The MDR1 gene as target, chemoprotectant and selectable marker in gene therapy, Adv. Pharmacology 46, 1-42 (1999)]. In connection with this, the quantitative characterization of its cell surface expression has prognostic significance in the clinical picture of many tumours, and it can orient the selection of the chemotherapy protocol. Even for these practical aspects it would be important to know the reasons, which are in the background of the variability of the degree of the Pgp expression, or the correlation with the clinical status [Nooter K. Sonneveld P.: Clinical Relevance of P-glycoprotein expression in haematological malignances, Leukemia Res., 18, 233-243 (1994); Bosch, I. and Crop, J.: P-glycoprotein multidrug resistance and cancer, Biochimica et Biophysica Acta 1288, 37-54 (1996)]. Among these the methodical sources of error [Sonneveld, P:: Prognostic significance and reversal of MDR in multiple myeloma, pp. 463-471, IN: Multidrug resistance in cancer cells. Molecular, biochemical, physiological and biological aspects, Ed.: Gupta S. and Tsuruo T., John Wiley and Sons (1996)], the uncertainty in connection with the clinical relevance of the degree of the Pgp expression (it is not known exactly that what level of Pgp expression can cause clinival multidrug resistance, or, the low level resistance can be independent from the Pgp), and the additive or alternative role of other resistance mechanisms [(Koshiyama, M., Yoshida, M., Fujii, H., Nanno, H., Hayashi, M. and Tauchi, K.: Expression of multidrug resistance-associated protein in endometrial carcinomas: correlation with clinicopathology and prognosis. Ann. Diagn. Pathol. 2:81-87. (1999); Osmak, M.: Multifactorial molecular mechanisms are involved in resistance of preirradiated human cervix carcinoma cells to cis-dichlorodiammineplatinum (II) and vincristine. Neoplasma 40:97-101 (1993); Michieli, M., Damiani, D., Ermacora, A., Masolini, P., Raspadori, D., Visani, G., Scheper, R. J. and Baccarani,: M. P-glycoprotein, lung resistance-related protein and multidrug resistance associated protein in de novo acute non-lymphocytic leukaemias: biological and clinical implications. Br. J. Haematol. 104:328-335 (1999)] can be mainly mentioned. The invention relates directly to the first two factors.

Many substances, which are called modulators or reverting agents [for instance Ca-channel blockers (verapamil), calmodulin inhibitors, steroids; the most effective Pgp inhibitors are the cyclosporin A, under clinical trial, the SDZ PSC 833 (a cyclosporin D derivative)] and the SDZ 280-466 peptid derivative [Friche, E. and Beck, W. T.: Molecular pharmacology of reversal of multidrug resistance and its clinical implications. pp. 362-374. In: Multidrug resistance in cancer cells. Molecular, biochemical, physiological and biological aspects E.d. Gupta S. and Tsuruo T. John Wiley & Sons (1996); Cornwell, M. M., Pastan, L and Gottesman, M. M.: Certain calcium channel blockers bind specifically to multidrug-resistant human KB carcinoma membrane vesicles and inhibit drug binding to P-glycoprotein. Journal of Biological Chemistry 262 2166-2170 (1987); Yusa, K., Naito, M. and Tsuruo, T.: Transport of P-glycoprotein-modifying agents by P-glycoprotein. pp. 331-344. In: Multidrug resistance in cancer cells. Molecular, biochemical, physiologycal and biological aspects. E.d. Gupta S. and Tsuruo T. John Wiley & Sons 199612-14)] can inhibit the transport of the substrates. It was demonstrated from most of these substances, that they increase the intracellular concentration of the cytostatics in the cells expressing Pgp, the most likely mechanism of which is the direct interaction with the Pgp, for example competition with the substrates for binding. Generally these modulators themselves are also transported. But at the same time the different substrates do not necessarily inhibit each other's transport. Since the Pgp molecules are expressed in the normal tissues, for example in the small intestines, in the bile-duct, in the renal tubules and in the capillaries of the brain [Gottesman, M. M., Hrycyna, C. A., Schoenlein, P. V., Germann, U. A. and Pastan, I.: Genetic analysis of the multidrug transporter, Annu. Rev. Genet. 29, 607-649 (1995); Thiebaut, F., Tsuruo, T., Hamada, H., Gottesman, M. M., Pastan, I., Willingham, M. C.: Immunohistochemical localization in normal tissues of different epitopes in the multidrug transport protein, P170: evidence for localization in brain capillaries and cross-reactivity of one antibody with a muscle protein. J. Histochem. Cytochem. 37:159-164 (1989)], when modulators are used, besides the tumorous tissue (which is frequently Pgp⁺) the concentration of the Pgp substrates can change in tissues expressing Pgp physiologically or in their neighbourhood, causing unexpected and undesired side effects [Schinkel, A. H., Mayer, U., Wagenaar, E., Mol, A. M., Deemter, L., Smit, J. J. M., Valk, M. A., Voordouw, A. C., Spits, H., Tellingen, O., Zjilmans, J. M., Fibbe, W. E. and Borst, P.: Normal viability and altered pharmacokinetics in mice lacking mdr1-type (drug-transporting) P-glycoproteins. Proc. Natl. Acad. Sci. USA 94 4028-4033 (1997); Miller, T. P., Grogan, T. M., Dalton, W. S., Spier, C. M., Scheper, R. J., Salmon, S. E. P-glycoprotein expression in malignant lymphoma and reversal of clinical drug resistance with chemotherapy plus high-dose verapamil. J. Clin. Oncol. 9 17-24. (1991); Friche, E. and Beck, W. T.: Molecular pharmacology of reversal of multidrug resistance and its clinical implications. pp. 362-374. In: Multidrug resistance in cancer cells. Molecular, biochemical, physiological and biological aspects E.d. Gupta S. and Tsuruo T. (1996); Pennock, G. D., Dalton, W. S., Roeske, W. R. Appleton, C. P., Mosley, K., Plezia, P., Miller, T. P., Salmon, S. E.: Systemic toxic effects associated with high-dose verapamil infusion and chemotherapy administration. J. Natl. Cancer Inst. 83 105-110 (1991)]. Effective, and in their side effects tolerable reverting agents represent a great chance for the cancer chemotherapy.

The exact mechanism for binding and removal of the substrates from the cell is not known yet, although detailed data are available in connection with the molecular mechanism and kinetic characteristics of the transport and modulability of many drugs, on the level of single cells and cell culture. But the details of that molecular recognition process, which accepts many hydrophobic compounds, are not known yet. Further refinement of the hydrophobicity criterium, supplementation with further structural parameters (at least one heterocyclic ring in the compounds, the relative distance of the electron donor groups, etc.) has not resulted yet in the discovery of such relations, which would show the essence of the substrate-enzyme relations that would make even the design of the substrates possible. At the same time the wide substrate spectrum has very specific characteristics: by the effect of the point mutations, causing the exchange of some amino acids, the efficacy of pumping is modified significantly, in a substrate-specific way. According to the most frequently cited theory of the pumping-mechanism, the hydrophobic molecules, accepted as substrates, are transported from the lipophilic medium, that is from the membrane, even before they reach the cytosol. This theory is supported most directly by the calcein-AM experiments of Sarkadi and co-workers—the compound is transported still as acethoxymethylesther (still in its “AM age”), and doesn't even have time to disintegrate by the effect of the esterases of the cytosol [Homolya, L., Hollö, Zs., Germann, U. A., Pastan, L, Gottesman, M. M. and Sarkadi, B.: Fluorescent cellular indicators are extruded by the multidrug resistance protein. Journal of Biological Chemistry 268 21493-21496 (1993); Hollö, Zs., Homolya, L., Davis, C. W. and Sarkadi, B.: Calcein accumulation as a fluorimetric functional assay of the multidrug trasporter. Biochimica et Biophysica Acta 1191 384-388 199420,21). Extrapolated from the detailed X-ray crystallographic data relating to other ABC transporters, the conformation change in the surrounding of the ATP binding site would expand to the substrate-binding regions, causing the translocation of the substrates from the on-site to the off-site.

Those experiments also refer to conformation changes, which detect the different binding properties of certain monoclonal antibodies, besides the substrate-saturation of the pump [Mechetner, E. B., Schott, B., Morse, S B., Stein, W., Druley, T., Davis, K. A., Tsuruo, T. and Roninson, I.: P-glycoprotein function involves conformational transitions detectable by differential immunoreactivity. Proceedings of the National Academy of Sciences, USA 94 12908-12913 (1997); Jachez B. Cianfriglia M. Loor F. Modulation of human P-glycoprotein epitope expression by temperature and/or resistance modulating agents. Anti-Cancer Drugs 5 655-665 (1994)]. One of these conformation-sensitive antibodies is the UIC2 antibody, the conformation events reflected by its substrate-dependent binding properties are in close relation with the present invention (?). Binding of the UIC2 antibodies increases 2-4-fold, for example in the presence of verapamil, vinblastin or cyclosporin A, etc. [Jachez B. Cianfriglia M. Loor F. Modulation of human P-glycoprotein epitope expression by temperature and/or resistance modulating agents. Anti-Cancer Drugs 5 655-665 (1994)]. This phenomenon is the so-called UIC2-shift, which is proposed by its discoverers to be used for the detection of the functional Pgp, or for the improvement of the detectability of small amount Pgp, for example in the U.S. Pat. No. 5,872,014. The methods and substances suitable for the detection of Pgp are described in the U.S. Pat. Nos. 5,994,088, 5,972,598, 5,830,697, 5,786,344, 5,773,280 és 5,434,075. Binding of other antibodies does not change, or hardly changes in the presence of modulators, substrates [Mechetner, E. B., Schott, B., Morse, S B., Stein, W., Druley, T., Davis, K. A., Tsuruo, T. and Roninson, I.: P-glycoprotein function involves conformational transitions detectable by differential immunoreactivity. Proceedings of the National Academy of Sciences, USA 94 12908-12913 (1997); Schinkel, A. H. Arceci, R. J. Smit, J. J. M, Wagenaar, E., Baas, F., Dolle, M., Tsuruo, T., Mechetner, E., Roninson, L B. and Borst, P.: Binding properties of monoclonal antibodies recognizing external epitopes of the human mdr1 P-glycoprotein. Int. J. Cancer 55 478484 (1993)]. Some of these conformation-sensitive antibodies can also be used as efficient inhibitors of the pump [Mechetner E B. Roninson I B.: Efficient inhibition of P-glycoprotein-mediated multidrug resistance with monoclonal antibody. Proceedings of the National Academy of Sciences, USA 89 5824-5828 (1992)]. It is an interesting characteristic of the UIC2 (Pgp-inhibitor) antibody, that it binds only to part of the cell surface Ppg molecules, the other binding sites—which are always accessible for example for the MRK16 antibodies—will become available for the UIC2 only in the presence of Pgp-substrates. (Meanwhile the binding constant of the antibody is also changing, but the change of the number of the binding sites is thought to be a dominant factor.) According to the (partly tentative) maps made from the epitopes recognized by different antibodies, the epitopes are formed by the extracellular members of the loops joining the 12 transmembrane helix. For example the MRK16 antibody probably recognizes the complex epitope formed by the #1 and #4 extracellular loops [Mechetner E B. Roninson I B.: Efficient inhibition of P-glycoprotein-mediated multidrug resistance with monoclonal antibody. Proceedings of the National Academy of Sciences, USA 89 5824-5828 (1992); Georges, E., Tsuruo, T. and Ling, V.: Topology of P-glycoprotein as determined by epitope mapping of MRK-16 monoclonal antibody. Journal of Biological Chemistry 268 1792 1798 (1993)]. In case of the UIC2, participation of the #1 loop in the antibody binding is documented [Schinkel, A. H. Arceci, R. J. Smit, J. J. M, Wagenaar, E., Baas, F., Dollé, M., Tsuruo, T., Mechetner, E., Roninson, L B. and Borst, P.: Binding properties of monoclonal antibodies recognizing external epitopes of the human mdr1 P-glycoprotein. Int. J. Cancer 55 478484 (1993)]. Our experimental data (described below), which form the basis of the present invention, are in close relation with the UIC2-shift phenomenon, described above.

### DISCLOSURE OF THE INVENTION

The invention is in relation to the phenomena detailed above, and summarized here. In case of the P-glycoprotein (Pgp), when modulators are used, 2-4 times more UIC2 antibodies bind to the cell surface Pgp-s (see the U.S. Pat. No.-s above). It is demonstrated here, that this increase of the UIC2 signal is accompanied by manyfold (even 60 times more) change in the binding of other antibodies, and this way the conformation change, which causes all these changes, can be detected much more sensitively. That is why this invention provides significant improvement of the so-called UIC2 procedure, which is based on the increase of the binding of the UIC2 antibody. Since some of the reverting agents (modulators) cause UIC2-shift, but doesn't cause change commensurable with the previously mentioned change, the application of the method according to the invention is not simply a sensibilization of the UIC2-shift method, but an other kind of technics, approach, which even uses inverse methodical steps for the detection of the change of the Pgp conformation (see below).

The invention is based on a surprising phenomenon: it was found, that the UIC2 antibody only slightly influenced the binding of the MM12.10 antibody [Romagnoli G. Poloni F. Flego M. Moretti F. Di Modugno F. Chersi A. Falasca G. Signoretti C. Castagna M. Cianfriglia M.: Epitope mapping of the monoclonal antibody MM 12.10 to external MDR 1 P-glycoprotein domain by synthetic peptide scanning and phage display technologies. Biol. Chem. 380 553-559 (1999)] (i.e. after binding of the UIC2 the MM 12.10 was still binding), but if the cell line expressing Pgp was treated with certain modulators/substrates, after the pre-incubation with UIC2 antibody the binding of the MM12.10 greatly decreased or stopped. This phenomenon was well reproducible with both the selected (KB-V1) and human mdr1 transfected (NIH 3T3 MDR1) cell lines (see FIG. 1 and 2). In case of incubation in reverse order the MM 12.10 has also effect to the binding of the UIC2 (prevents it), but this effect does not prevail in a modulator dependent way (see FIG. 5).

Not all modulators behave similarly (see FIG. 1 and 2). While cyclosporin A and vinblastine results in the phenomenon mentioned above, the verapamil even in effective dose (which causes nearly maximal Pgp function inhibition) doesn't cause such an effect. Many other reverting agents and substrates were also compared. Two extremely different type of modulators/substrates can be distinguished, one of which accomplishes the conformation change, which prevents binding of MM12.10 after UIC2, while the other doesn't. The first group: Cyclosporin A (CSA), PSC 833, vinblastine and valinomycine, the second group: verapamil, Tween 80, nifedipine, progesterone, actinomycine D, prazosin and gramicidin. Fluorescence resonance energy transfer and confocal microscopic measurements were also carried out, using the two antibodies: these preliminary experiments of ours demonstrated, that in the absence of competition the two different antibodies do not bind to the same Pgp molecule. Our aforementioned data indicate, that two different Pgp populations can be distinguished on the same cell.

The correlation among the results of the ACT, UIC2-shift and stain-accumulation tests was investigated by simultaneous carrying out of the three procedures (see FIG. 3). The R_{competition}, R_{activity} and R_{shift} parameters, i.e. the ratios of the average fluorescence intensities measured in the presence and absence of the competing antibodies, were plotted against each other. The CSA-induced R_{competition} was 30-60-fold, against the 1,5-2-fold R_{shift} values. Modulation of the Pgp function was about the same in these experiments. The function-modulation was always accompanied by UIC2-shift, whether the indicator was daunorubicin (DNR) or calcein. At the same time, the function-modulation didn't always result in great R_{competition} values in ACT.

Since the ATP depletion is causing known increase in UIC2 reactivity [Mechetner E B. Roninson I B.: Efficient inhibition of P-glycoprotein-mediated multidrug resistance with monoclonal antibody. Proceedings of the National Academy of Sciences, USA 89 5824-5828 (1992)], the effect of oligomicin or sodium-azide and 2-deoxy-D-glucose treatment for the ACT results was also investigated. As it can be seen on FIG. 4, the ATP depletion gave big R_{competition} value in the ACT too, similarly to CSA.

Using the possible combinations of the anti-Pgp antibodies, one after the other, on live, Pgp positive cells, it can be observed with other antibody-pairs too, that there is a significant difference in the binding of the antibody added secondly, in the presence or absence of modulators (FIG. 5). For example, adding first the UIC2 antibodies, binding of the MRK16 (see the U.S. Pat. Nos. above) or the MM8.15 [Cianfriglia M. Loor F. Modulation of human P-glycoprotein epitope expression by temperature and/or resistance modulating agents. Anti-Cancer Drugs 5 655-665 (1994)] antibodies, added secondly, was not drastically influenced (50% maximum), when modulators, substrates were not present. But if these are present, then UIC2 totally inhibited binding any of the four antibodies mentioned above.

The invention is described in details. The attached drawings are summarized hereunder:

### BRIEF DESCRIPTION OF THE DRAWINGS

FIG. 1 shows the effect of the treatment with reverting agents to the binding ratio of the Pgp-specific antibodies on KB-V1 cells (flow cytometry curves).

FIG. 2 shows the inhibitory effect of the UIC2 antibody to the binding of FITC-MM12.10 antibody, in the presence of different reverting agents, on NIH3T3 MDR1 cells (flow cytometry curves).

FIG. 3 shows the investigation of the correlation between the ACT (R_{competition}, for the UIC2/FITC-MM12.10 antibody pairs), the UIC2-shift (R_{shift}) and the Pgp function (R_{activity}). Each symbol represents independent experimental data.

FIG. 4 shows the effect of the ATP-depleting agents on NIH3T3-mdr cells (ACT).

FIG. 5 shows the results of the ACT investigations on NIH3T3 cells, in case of different antibody combinations.

FIG. 6 shows the effect of the CD4-ligation to the ratio of the binding of two antibodies.

### BEST MODE OF CARRYING OUT THE INVENTION

The invention describes an antibody competition method for the detection of the conformation changes of cell surface receptors, pumps, proteins, including the P-glycoprotein and CD4. According to the method of the invention, two antibodies and/or ligands are attached to receptors, pumps and/or proteins, recognized by them, and from the difference of the binding, the conformation status, or the change of the conformation status of the receptors or antigens is concluded.

In a preferred embodiment of the invention other receptors, antigens, pumps are used on cell surface or membrane preparatum, or on isolated proteins or artificially produced proteins, for the detection of the conformation status of the proteins, or the change of the latter, or for their binding to ligands, antibodies, substrates, modulators.

In a preferred embodiment the invention is used for P-glycoprotein, or other pumps in connection with multidrug transport on membrane preparates, or for isolated proteins or artificially produced proteins for the detection of the conformation state of the proteins or for the change of the latter, or for the detection of its binding to ligands, antibodies, substrates and modulators.

In a practical implementation the invention is used for the detection of the interaction, binding of the P-glycoprotein with ligands (i.e. substrates, reverting-chemosensitizing-mudulating agents).

The method of the invention, besides the uses mentioned above, provides a very exact and followable method for the development and/or detection of new reverting (=chemosensitising or modulating) agents. It is very useful in the estimation of the modulateability of the resistance with reverting (=chemosensitising or modulating) agent in case of cell surface the P-glycoprotein pump protein, which is frequently behind the multidrug resistance, both on cell lines and cells originating from humans, for basic research, diagnostics and therapy alike.

The method of the invention can generally be used for any kind of cell surface pump, which is frequently behind the multidrug resistance. The method can advantageously be used for the cell surface P-glycoprotein pump, and the invention is mainly described in connection with this. However, it must be obvious for the experts, that the scope of the appended claims is not limited to the use of the protein mentioned above, but it can be used for any other cell surface pump and receptor, ligand-binding protein.

According to the method of the invention any antibody or ligand, known for the experts, can be used, which reacts with the receptors, antigens, pumps on the cell surface, membrane preparate, isolated or artificial proteins. Not just the antibodies or ligands, but their different parts, prepared by chemical or genetic engineering methods, can also be used.

According to one of the most preferred embodiment of the invention two monoclonal or polyclonal antibodies or ligands, labelled with different fluorescing stains, are attached to the Pgp epitopes recognized by them, simultaneously or sequentially, and their binding is measured, preferably with flow cytometry or other method suitable for the detection of the fluorescence, in order to conclude on the conformation state of the receptor or antigen recognized by the antibodies or ligands from the ratio of the two signals, or from the change of the ratio.

An embodiment of the present invention is the use of receptors, antigens, pumps on the cell surface, membrane preparate, isolated or artificial proteins for the detection of the conformation state of the protein, or change of the latter, or for the detection of the binding of the protein to ligands, antibodies, substrates, modulators, which is based on some kind of indirect (biotinilating, use of secondary antibodies, etc.) fluorescence signal of the bound antibodies.

According to another preferred embodiment of the invention detection of the binding of receptors, antigens, pumps on the cell surface or membrane preparate, isolated or artificial proteins is carried out by direct fluorescence labelling and detection of the ligands specifically recognizing the proteins mentioned above.

In the method of the invention practically such agents are used, in which the two ligands and antibodies, bound to the protein and fluorescently labelled, compete with each other for a binding site, or bind to overlapping binding sites, or allosterically influence each other's binding.

According to a preferred embodiment of the method of the invention only one of the antibody or ligandum, used for the detection of the conformation change is labelled, the other is unlabelled, and competes with the labelled agent, or influences its binding allosterically.

According to an especially preferred embodiment of the method of the invention, a mixture of antibodies or ligands, labelled with two different fluorescent stains, are given to cells grown or stored on microtiter plates, and different agents are added to the wells, and the ratio of the binding of the two different antibodies or ligands is detected by appropriate means, and study or measure the effect of the added agents to the ratio of the bound fluorescent molecules.

An example of the method mentioned above is a cell line or ex vivo cell preparate, expressing any P-glycoprotein, grown or stored in the microtiter plate, different modulators (reverting agents, etc.), or substances presumably having this effect, or different concentration solutions thereof are added to to the wells, and the mixture of the two different (for example fluorescing in green and red) anti-Pgp antibodies is measured, or these are used sequentially, and (after washing off the not-bound antibodies) the two fluorescent signals are detected, and their ratio is determined for the wells, and this way the substance, having the greatest influence on this ratio, is selected.

Another example of the method mentioned above a cell or cell line that expresses CD4, grown or stored on microtiter plates, different CD4-binding ligands (aurintricarboxilic acid derivatives, genetically manipulated variants of gp120, etc.), or substances presumably having this effect, or different concentration solutions thereof are added to to the wells, and the mixture of the two different (for example fluorescing in green and red) anti-CD4 antibodies is measured, or these are used sequentially, and (after washing off the cells) the two fluorescent signals are detected, and their ratio is determined for the wells, and this way the substance, having the greatest influence on this ratio, is selected.

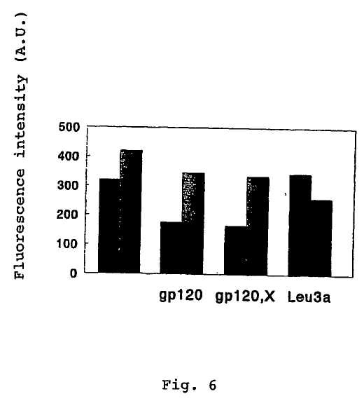

Another preferred embodiment of the method of the invention is the application in case of CD4 molecules. The CD4 molecule is having a role in the immunological recognition too, but on the limfocites, expressing this molecule enables the HIV virus (causing AIDS) to enter CD4-positive cells, by the binding of HIV to a known epitope of CD4. Meanwhile CD4 undergoes a conformation change [Szabó, G. jr., Pine, P. S., Weaver, J. L., Kasari, M., Aszalos, A.: Epitope mapping by photobleaching fluorescence resonance energy transfer measurements using a laser scanning microscope system. Biophys. J.. 61, 661-670 (1992); Szabó, G., jr., Pine, P. S., Weaver, J. L., Rao, P. E., Aszalos, A.: CD4 changes conformation upon ligandum binding. J. Immunol.. 149, 3596-3604 (1992); Szabó, G. Jr., Pine, P. S., Weaver, J. L., Kasari, M., Aszalos, A.: Cross-linking of CD4 in a TcR/CD3-juxtaposed inhibitory state: a pFRET study. Biophys. J.,. 68, 1170-1176 (1995); Yachou A and Sekaly R P.: Binding of soluble recombinant HN envelope glycoprotein, rgp 120, induces conformational changes in the cellular membrane-anchored CD4 molecule. Biochemical and Biophysical Research Communications 265:428-433 (1999)]. This change in the conformation reflects also in the change of the binding ratio of the two antibodies bound to other epitopes of the molecule (FIG. 6). This way by simultaneously measuring the cell surface binding of the two antibodies, the change of the CD4 conformation can be detected, and the specific ligandum binding of CD4 can be recognized.

Therefore with the method of the invention it is possible to search and screen for new natural and artificial ligands, which were not known for binding CD4, for the development of HIV therapy. The method also provides the possibility for the fluorescent measurements based on the theory mentioned above on CD4 molecules or on fragments thereof, carried by live or dead cells, or isolated or artificially produced, and the discovery and/or isolation of viruses, which are mutated in the different CD4 binding, or altered for other reasons.

The method of the invention is significantly different from the methods developped earlier, and that is why it is more efficient. In an example of the method of the invention, relating to Pgp, competition of the UIC2 antibody (on CSA-treated cells, in the second step with FITC-labelled antibody) manifested even in 60-fold change of the fluorescence intensities measured by flow citometry. This way the sensitivity of the detection of the conformation changes increases, compared to the UIC2 shift. Another difference, compared to the UIC2 shift, is the fact that certain reverting agents proved to be efficient (CSA, vinblastin, valinomycin), while others (verapamil, Tween 80) not. This way the so far unknown classification, categorization of the reverting agents, separation of the different reverting agents becomes possible. The facts mentioned above refer to the substantial difference between the UIC2-shift test and the antibody competition test (“ACT”).

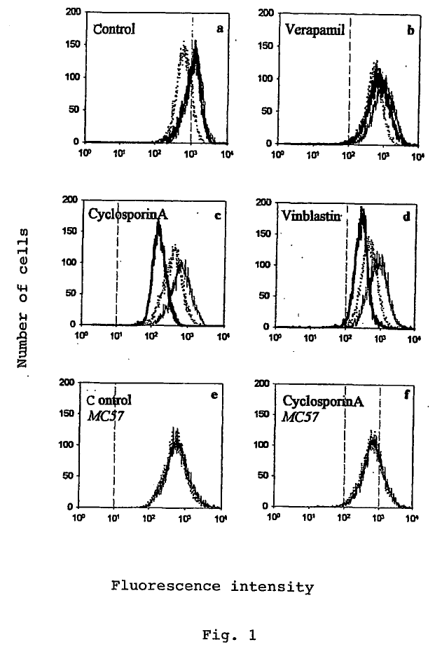

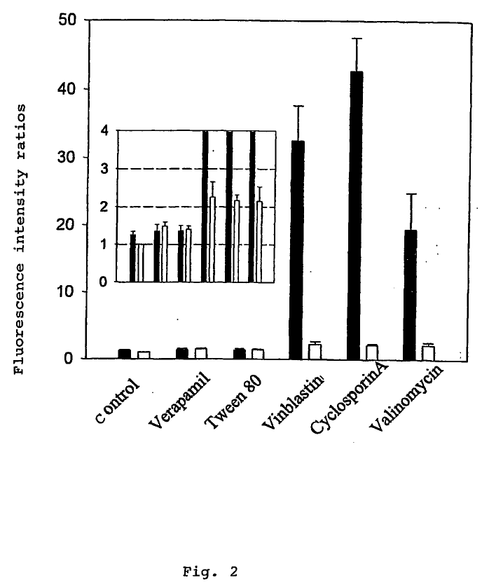

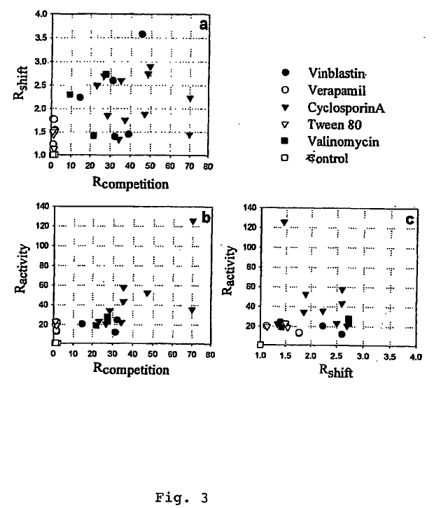

It is characteristic of the ratio of the UIC2-shift test, known from the literature (see the U.S. Pat. Nos. above) and the antibody competition test (ACT), that in the former, when using modulators, generally 2-4 times more UIC2 antibody bind to the cell surface Pgp-s. As it can be seen from the data of FIG. 1 and 2, this increase in the UIC2-signal is accompanied by manyfold (even 60-fold) change in the binding of other antibodies (for example MM 12.10), and this way the conformation change, causing all these thing, can be detected much more sensitively. This means, that our ACT method, at the same time means the significant development of the so-called UIC2-shift method, which is based on the increase of the binding of the UIC2 antibody. Since some of the reverting agents cause UIC2-shift, but in the ratio of the binding of the two antibodies do not cause a change commensurable to the former (see FIG. 3), the use of this ACT is not simply the sensitization of the UIC2-shift technics, but a different technics, approach for the detection of the changes of the Pgp conformation.

The stain-accumulation tests, like measuring the accumulation of R123 [Lee, J S., Paull,.K., Alvarez, M.: Rhodamine efflux patterns predict P-glycoprotein substrates in the National Cancer Institute Drug Screen. Mol. Pharmacol. 46: 627-638 (1994)], or recently the calcein-AM [Homolya, L., Holló, Zs., Germann, U. A., Pastan, L, Gottesman, M. M. and Sarkadi, B.: Fluorescent cellular indicators are extruded by the multidrug resistance protein. Journal of Biological Chemistry 268 21493-21496 (1993); Holló, Zs., Homolya, L., Davis, C. W. and Sarkadi, B.: Calcein accumulation as a fluorimetric functional assay of the multidrug trasporter. Biochimica et Biophysica Acta 1191 384-388 199420,21); Homolya, L., Holló, Zs., Müller, M., Mechetner, E. B., Sarkadi, B.: A new method for a quantitative assessment of P-glycoprotein-related multidrug resistance in tumour cells. Br. J. Cancer 73 849-855 (1996); Holló, Zs., Homolya, L., Hegedüs, T., Müller, M., Szakács, G., Jakab, K., Antal, F., Sarkadi, B.: Parallel functional and immunological detection of human multidrug resistance proteins, P-glycoprotein and MRP1, Anticancer Res. 18 2981] on living cells is a very effective method of the measurement of Pgp function, which can be used for diagnosing the presence of other pumps. But their significant disadvantage is, that they can be used only on living cells optimally at the site of sampling. But at the same time a great advantage of the method of the invention, or the measuring possibilities based on it that they can be carried out simply and with absolute certainty any time after fixation of the cells. But the method can be carried out even that way, that the cells are fixed before labelling with the antibodies. In this case binding of the UIC2 antibody indicates the Pgp molecules in UIC2-binding conformation on the surface of the cells, while binding of the other antibody (for example MM12.10) indicates those Pgp-s, which would be labellable with UIC2 only in the presence of modulators (chemosensitizing agents, substrates).That is, in this case we get a test without modulator treatment, which results in more information than the UIC2-shift assay.

[As to the effect of influencing the UIC2 binding of different fixation procedures, see: Katalin Goda, Henrietta Nagy, László Bene, Margit Balázs, Robert Arceci, Eugene Mechetner, Gábor Szabó: Conformational heterogeneity of P-glycoprotein. Cancer Detection and Prevention, 24(5) 415 (2000)].

The invention is illustrated in details by the following examples. But it must be obvious for the experts, that it can be realized with steps and materials different from the described, and the scope of the appended claims is not limited to the examples.

Materials and Methods

Cell Lines

KB-3-1 (Pgp⁻) and the slightly resistant KB-8-5, and the strongly resistant KB-V 1 (Pgp⁺) human epidermal carcinoma cell lines, NIH3T3 MDR1 transfected cell line. These cell lines are from the laboratory of Dr. Michael Gottesman (NIH, Bethesda) [see for example: Stability and covalent modification of P-glycoprotein in multidrug-resistant KB cells. Richert N D, Aldwin L, Nitecki D, Gottesman M M, Pastan I.: Biochemistry 27 20 7607-13 (1988 okt. 4); ATP-binding properties of P glycoprotein from multidrug-resistant KB cells. Cornwell M M, Tsuruo T, Gottesman M M, Pastan I. FASEB J.: 1 51-4 (1987 július 1)].

They can be cultured as monolayer cultures at 37° C., with 5% CO2, in Dulbecco's Minimal Essential Medium (with the presence of 10% fetal calf serum, 25 μg/ml gentamicin and 2 mM L-glutamine). The drug resistant cells are to be cultivated continously in the presence of the suitable drug (180 nM vinblastin (KB-V 1), 25 nM colchicine (KB-8-5), and 690 nM doxorubicin (NIH3T3 MDR1)). Two-three days before the experiments the cells were trypsinized, and were incubated in drug-free medium until use.

Drugs

Verapamil, vinblastine, cyclosporin A, actinomycin D, prazosin, progesterone, valinomycin, nifedipine, gramicidin: Sigma-Aldrich (Budapest). Tween 80: SERVA (Heidelberg, Germany). Modulator concentrations used for the treatment of cells: 50 μM verapamil, 10 μM cyclosporin A, 70 μM vinblastin, 0.002% Tween 80, 10 μM valinomycin, 5 μM actinomycin D, 10 μM prazosin, 10 μM progesterone, 50 μM nifedipine, 5 μM gramycidin. The concentration of ATP-depleting agents: 5 μM oligomycin and 5 mM sodium azide (used with 5 mM 2-deoxy-D-glucose). Cell culture mediums and additives: Sigma. 6-(fluorescein-5-carboxamido)hexanoic acid succinimidyl ester (5-sFx) and fluorescein-5-isothiocyanate (FITC): Molecular Probes (Eugene, Oreg.). All other chemicals were of analytical grade (Sigma). Description of the method of FITC conjugation, used here can be found in the literature [DePetris S.: Methods in Membrane Biology Vol. 9. (Ed. Korn, E. D.) Plenum Press, New York, pp.1-201 (1978); Spack, E G. Jr. Packard, B. Wier, M L. Edidin, M.: Hydrophobic adsorption chromatography to reduce nonspecific staining by rhodamine-labeled antibodies. Anal. Biochem. 158 233-237 (1986)].

Description of the ACT Method

The cells were trypsinized as usual, washed twice with PBS (phosphate buffered saline, pH 7,4). 10⁶ cells/1 ml PBS (plus 8 mM glucose), pre-incubated for 20 minutes at 37° C. with different drugs, modulators, and without washing the cells, the first (“A”) monoclonal antibody was added to the samples. After a further 30 minutes incubation (at 37° C.) an FITC or 5-sFx conjugated second (“B”) antibody was added to the samples (again without washing the cells), and were incubated at 37° C. for another 30 minutes. In case of the UIC2-shift test the cells were incubated with UIC2 antibody, washed twice, and incubated with goat anti-mouse IgG2a FITC conjugate (F/P=4,3, Sigma) on ice in 100 μl PBS for 45 minutes. The samples washed twice were resuspended in 500 μl PBS, and analyzed in flow cytometer. The antibody concentrations used were: 8 μg/ml MRK16, MC57, MM8.15, MM12.10 and 10 μg/ml UIC2. Daunorubicine (DNR) and calcein accumulation tests: see Goda, K. et al.: Practical guide to physical analysis of cell surface receptors, Ed.: Krasznai, Z. and Mátyus, L., pp. 110-125 (1998); Homolya, L., Holló, Zs., Germann, U. A., Pastan, L, Gottesman, M. M. and Sarkadi, B.: Fluorescent cellular indicators are extruded by the multidrug resistance protein. Journal of Biological Chemistry 268 21493-21496 (1993); Holló, Zs., Homolya, L., Davis, C. W. and Sarkadi, B.: Calcein accumulation as a fluorimetric functional assay of the multidrug trasporter. Biochimica et Biophysica Acta 1191 384-388 199420,21.

The average fluorescence intensities per cell were determined by Becton Dickinson FACScan (Mountain View, Calif.). Dead cells were excluded from the analysis by propidium-iodate staining. The fluorescence signals were plotted on logarithmic scale, the two-level analysis was carried out by BDIS CellQuest and FloWin softwares (SoftFlow Ltd., Pécs, Hungary).

Antibodies and Labelling UIC2 [Mechetner E B. Roninson I B.: Efficient inhibition of P-glycoprotein-mediated multidrug resistance with monoclonal antibody. Proceedings of the National Academy of Sciences, USA 89 5824-5828 (1992)], MM12.10 [Romagnoli G. Poloni F. Flego M. Moretti F. Di Modugno F. Chersi A. Falasca G. Signoretti C. Castagna M. Cianfriglia M.: Epitope mapping of the monoclonal antibody MM 12.10 to external MDR 1 P-glycoprotein domain by synthetic peptide scanning and phage display technologies. Biol. Chem. 380 553-559 (1999)] MRK16 [Gottesman, M. M., Hrycyna, C. A., Schoenlein, P. V., Germann, U. A. and Pastan, I.: Genetic analysis of the multidrug transporter, Annu. Rev. Genet. 29, 607-649 (1995)] antibodies. The 5-sFx [6-(fluorescein-5-carboxamido)-hexanoic acid-succinimid-ester] and X-TRITC (rhodamine red™-X succinimid-ester) labellings were carried out according to standard methods.

### EXAMPLE 1

In clinical, mainly hemoproliferative pathographies the study of the Pgp expression with the ACT method of the invention, or the detection of functional Pgp on multidrug resistant cells, or cells suspected to be resistant, for human diagnostic purposes. For the latter the significant advantage of the method, against the methods measuring the stain pumping on living cells, (calcein-AM or R123 assay, stc.) is that the cells labelled after fixing can be stored for a long time, can be transported, and the samples can be analyzed even far from their place of origin, or this is possible even with the collected samples. According to our experience our ACT method is more sensitive than the UIC2 test (more detailed comparison of the UIC2, calcein and ACT test see below).

In connection with the therapy of tumours, the question frequently arises, which is difficult to answer, whether the cells are multidrug resistant, which fact influences the selection of the treatment protocol. For the determination of the drug resistance, stain accumulation tests or antibody binding test can be carried out. The disadvantage of the former, that it gives reliable results only with fresh samples. The disadvantage of the latter, that it detects only the presence of the pump proteins. By comparing the UIC binding of Pgp, which is most frequently in the background of multidrug resistance, before and after CSA, detection of functional Pgp molecules is possible. A more sensitve variation of this is ACT.

The single cell suspension originating from a tumor tissue, for example leukemia cells are washed twice with PBS (phosphate buffered saline, pH 7,4). 10⁶ cells/1 ml PBS (+8 mM glucose) cell concentration is set, and after pretreating with 10 μM CSA for 10 minutes, the sample is divided in two aliquots. Than, one of the aliquots (I) is preincubated with the first (“A”) monoclonal antibody in 10-100 μg/ml concentration. Antibody is not added to the second aliquot (II), instead PBS is added. After 30 minutes incubation (37° C.) the FITC or 5-sFx conjugated second (“B”) antibody is added to the sample in 1-100 μg/ml concentration (without washing the cells), and is further incubated at 37° C. for another 30 minutes. The samples washed twice with PBS are resuspended in 1 ml PBS, and analyzed in flow cytometer. The average fluorescence intensities per cell were determined after the exclusion of the dead cells by propidium-iodate staining. The ACT value is calculated by the ratio of the average fluorescence intensity of the samples I and II, i.e. ACT=II/I. The protocol is the same with the fixed cells. In the latter case, following labelling with antibodies, the samples are fixed in 1% formaldehyde solution (prepared in PBS) at 4° C. for half an hour, and then the cells can be stored in this same formaldehyde solution at 4° C. (at least for a few weeks). “A” antibody: UIC2, “B” antibody: MM1210 or MRK16, all the three used in saturation concentration.

The former protocol can also be used for predicting the efficacy of the designed cytostatic drug combination. In this case an aliquot of the native cells with the drug concentration, that can be expected at the place of origin (i.e. blood) are incubated with the drugs (instead of CSA), and the ACT test is carried out (further dividing the sample according to the protocol described above). The ratio of the average fluorescence values of the two samples prepared this way (after substracting the autofluorescence values) gives the ACT value: ACT=II/I, which, if it is close to 0, means the interaction of the drug or the drug combination with Pgp, that is the possible inefficacy of the therapy, or even the multidrug resistance modulation feature of some of its components, with the given sample or patient, and the value around 0.5 (0.2-0.9), or the identity of this value measured in the absence of drugs, with the calculated ACT means the absence of the interaction of Pgp and the used drugs, that is the potential efficacy of the planned treatment.

### EXAMPLE 2

Quick and quantitative assay for the detection of the changes of the conformation-topologic relations, occurring during Pgp modulation. This test is a new means for searching more efficient modulators, and applicable for the detection and investigation of the conformation changes occuring during the interaction of Pgp and different reverting agents and substrates. It can be used for screening, testing of reverting agents, or for the determination of these kind of effects in case of compound known before. Our ACT method can only be used for the detection of the most effective reverting agents, against the UIC2 shift method, which can detect even the verapamil effect. That is selective testing of the reverting agents with strategies different from the previous agents, and selective testing of the most effective agents, resulting in the biggest conformation change can be done.

The cultures of NIH 3T3 MDR (or other cell line, transfected with human MDR1) are used [Homolya L., HollóZ., Germann U A, Pastan I., Gottesman M M., Sarkadi B.: Fluorescent cellular indicators are extruded by the multidrug resistance protein. Journal of Biological Chemistry 268, 29 21439-6 (1993. okt. 15)]. These cells can generally be cultured in monolayer cultures, at 37° C., with 5% CO₂, in Dulbecco's Minimal Essential Medium (in the presence of 10% fetal calf serum, 25 μg/ml gentamycin and 2 mM L-glutamine). The drug resistant cells are to be cultivated continously in the presence of the suitable drug (which is optimal to the cells given, in our case for example in the presence of 690 nM doxorubicin). Two-three days before the experiments the cells are trypsinized, and were incubated in drug-free medium until use. The cells are washed twice with PBS (+8 mM glucose) pre-incubated for 20 minutes at 37° C. with different drugs, modulators. Without washing the cells, the first (“A”) monoclonal antibody was added to the samples. After a further 30 minutes incubation (at 37° C.) an FITC or 5-sFx conjugated second (“B”) antibody was added to the samples (again without washing the cells), and were incubated at 37° C. for 30 minutes. The samples washed twice are resuspended as usual, for example in 500 μl PBS, and analyzed in flow cytometer. The applied antibody concentrations: 8 μg/ml MRK16, MC57, MM8.15, MM12.10 and 10 μg/ml UIC2. It is generally suitable to use the antibodies in saturation concentration, especially in case of the first (“A”) antibody. The average fluorescence intensities per cell were defined. Dead cells were excluded from the analysis by propidium-iodate staining. Treatments can also be carried out in microtiter plates, the washing-centrifugation steps can be solved in any laboratory, or these steps can also be automatized. Instead of flow cytometric evaluation, the fluorescence intensity per wells can be read with fluorescent microtiter plates after three PBS washing and the last antibody incubation, and using these values the ACT values per wells can be calculated.

### EXAMPLE 3

### Method for the Verification and Study of the so-called Alternative Conformation Statuses of the Pgp [for Example: Mechetner, E. B., Schott, B., Morse, S B., Stein, W., Druley, T., Davis, K. A., Tsuruo, T. and Roninson, I.: P-glycoprotein Function Involves Conformational Transitions Detectable by Differential Immunoreactivity. Proceedings of the National Academy of Sciences, USA 94 12908-12913 (1997)]

If both antibodies, i.e. UIC2 and MRK16 are labelled, one with green (for example FITC) the other with red (for example TRITC or PE) fluorescent stains, and the ratio of the two different colour fluorescence is measured with flow cytometry or fluorescent microtiter plate reader (or even with microscope based instruments), then more delicate deviations can also be detected with this method of increased sensitivity. (In this case for example the UIC2 sign increases, the MRK16 sign decreases by the effect of the modulators). Since the extent of the fluorescent resonance energy transfer results in less than 20% intensity changes between UIC2 and MM 12.10 on living cells, or membrane preparate, and changes at most in this order of magnitude can be expected, as a result of this effect this factor doesn't complicate the “two colour” application of our method.

### EXAMPLE 4

### The Effect of Reverting Agents to the Binding Ratio of Pgp-specific Antibodies (on ACT KB-V1 Cells) (FIG. 1) of Pgp-specific Antibodies (on ACT KB-V1 Cells) (FIG. 1) of Pgp-specific Antibodies (on ACT KB-V1 Cells) (FIG. 1)

The cells were pretreated with modulators (CSA or verapamil, b-10 d, f), or were incubated without pretreatment (control, a, e), at 37° C., for 30 minutes. The cells are then treated with the first (“A”) antibody (incubation with or without UIC2, MM8.1 or MC57) for 30 minutes, and the cells are labelled with FITC-MM12.10 (“B”) antibody The curves are flow cytometry histograms from representative measurement each. Continous line: control without “A”; bold line: “A”=UIC2; dotted line: “A”=MM8.15 (a-d) or MC57 (e-f). Binding of the “A” antibodies was verified in two independent experiments, with indirect immunfluorescence.

### EXAMPLE 5

### Data of Flow Cytometry Experiments (FIG. 2)

The UIC2 antibody inhibits binding of FITC-MM12.10 antibody in the presence of reverting agents, on NIH3T3 MDR1 cells. The ratio of average FITC MM12.10 fluorescence intensities, with and without UIC2 pretreatment give the R_{competition} values (black columns). Cells were pretreated for 20 minutes with reverting agents, and incubated for further 20 minutes with UIC2 antibody, finally the FITC-MM12.10 antibody was used (further 30 minutes incubation). Cells were not washed between the incubation steps. Concentration of the reverting agents: Verapamil (50 μM), Tween 80 (0,002%), Vinblastin (70 μM), Cyclosporin A (10 μM), Valinomycin (10 μM). UIC2-shift was detected by indirect immunfluorescence; the white columns show the ratio of the average fluorescence intensities detected with and without the reverting treatments (R_{shift}). On the insert figure the same UIC2-shift data are plotted on magnified scale. The average of 4-6 independent measurements (±SEM) are shown.

### EXAMPLE 6

### Simultaneous ACT (FIG. 3)

Investigation of the correlation between (R_{competition UIC}2/FITC-MM12.10 antibody pair), UIC2-shift (R_{shift} and Pgp function (R_{activity}). Each symbol represents independent experimental data.

### EXAMPLE 7

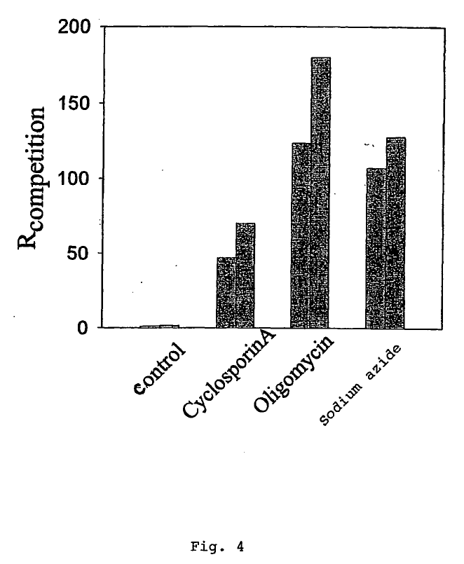

The effect of ATP-depleting agents was investigated on NIH3T3-mdr cells, with the ACT method of the invention. The results the investigation are shown on FIG. 4.

### EXAMPLE 8

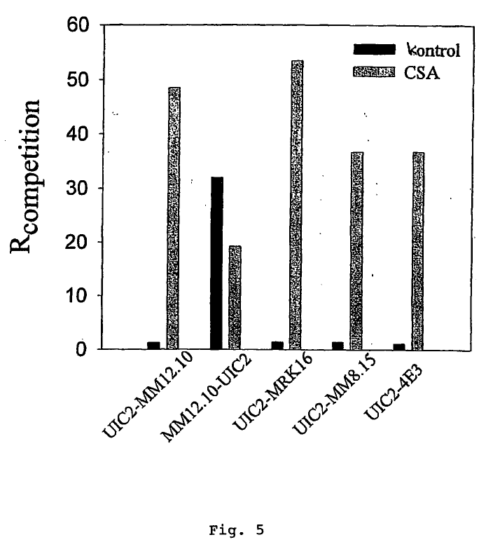

ACT tests were carried out on NIH3T3 MDR1 cells, with different antibody combinations. Following UIC2 or MM12.10 (not conjugated with stain) pretreatment the cells are labelled with FITC or 5sFx conjugated MM12.10, UIC2, MRK16 and MM18.15 antibodies. Before adding the antibodies the cells are pretreated with CSA (grey columns) or not (black columns). The results of two independent experiments are shown on FIG. 5.

### EXAMPLE 9

The effect of CD4-ligation to the ratio of the binding of two antibodies was investigated. After gp120, gp120+anti-gp120, or Leu3a pretreatment the cell surface receptors of the CD4-positive human lymphocites were simultaneously labelled with OKT4E-FITC (black columns) and OKT4-PE (grey columns) antibodies, and the average FITC (green) and PE (red) fluorescence intensities per cell were determined by flow cytometry. The results of two independent experiments are shown on FIG. 6.

What is claimed is:

1. A method of detecting the presence and/or conformation change of cell surface receptors, pumps or proteins comprising binding at least two different antibodies, and optionally a ligand to the receptors, pumps and/or proteins recognized by the antibodies, and the changes of the antibody binding is measured.

2. A method according to claim 1 wherein the antibody is a monoclonal or polyclonal antibody, or a fragment thereof.

3. A method according to claim 1 wherein monoclonal antibody is used.

4. A method according to any of the claims 1-3 wherein one of the antibodies is UIC2 and/or its fragment.

5. A method according to any of the claims 1-4 wherein the protein is P-glycoprotein.

6. A method according to any of the claims 1-4 wherein the protein is CD4 cell surface protein.

7. A method according to any of the claims 1-6 wherein the ligand is substrate or reverting chemosynthesizing and/or modulating agent.

8. A method according to claim 7 wherein the substrate is Cyclosporine A, PSC833, vinblastin or valinomycine.

9. A method according to claim 7 wherein the substrate is verapamil, Tween 80, nifedipine, progesterone, actinomycine D, prazosin or gramicidin.

10. A method according to claim 4 wherein the second antibody is MM12.10, MC57, MM8.15 or MRK16.

11. A method according to claim 6 wherein the ligand is anti-CD4 antibody, any component of the HIV virus or aurintricarboxilic acid.

12. A method according to claim 11 wherein the HIV virus component is the naturally occurring or artificially created form of the gp120 protein.

13. Method for the determination of cytostatic drug combination, effective in case of multidrug resistance wherein the native cells are combined in vitro vith the drug or drug combination to be tested, and the sample is tested with the method according to claim 1.

14. Method according to claim 13 wherein the method according to claim 1 is carried out with Cyclosporin A modulators.

15. Method for the detection of the efficiency of multidrug-resistance modulators wherein the method according to claim 1 is carried out in the presence of known modulator or a modulator to be tested, and the resulting activity values are compared.

16. Method according to claim 15 wherein Cyclosporin A, PSC 833, vinblastin or valinomycine is used in the controll tests.

17. Method according to any of the claims 1-16 wherein at least one of the antibodies is labelled.

18. Method according to claim 17 wherein at least one of the antibodies is labelled with fluorescent stain.

19. Method according to claim 17 or 18 wherein both antibodies are labelled.

20. Method according to claim 19 wherein the two antibodies are labelled with two different fluorescent stains.

21. Method according to any of the claims 17-20 wherein the determination is made by flow cytometry or with other method, suitable for the determination of fluorescence.

22. Method according to claim 21 wherein the fluorescence microtiter plate method is used.

23. Method according to any of the claims 1-21 wherein the cells labelled with antibodies are fixed between labelling and measurement of binding.

24. Method according to any of the claims 1-21 wherein the cells are fixed before labelling with the antibodies.

25. Diagnostic kit for the detection of the presence and conformation change of receptors, pumps and proteins, for the determination of the efficient drug combination in case of multidrug resistance and/or for the detection of the efficiency of the modulators comprising
a first antibody which is able to detect the receptors, pumps or proteins,
a second antibody which is able to detect the receptors, pumps or proteins,
optionally other ligands,
and a system, which provides a system for the detection of binding.
